(19) United States
(12) Patent Application Publication
(10) Pub. No.: US 2020/0341013 A1
(43) Pub. Date: Oct. 29, 2020

(54) MODIFICATION TO STAGING GUIDELINES FOR IMPROVED ACUTE KIDNEY INJURY (AKI) RECOVERY DETECTION

(71) Applicant: KONINKLIJKE PHILIPS N.V., Eindhoven (NL)

(72) Inventors: ERINA GHOSH, BOSTON, MA (US); LARRY JAMES ESHELMAN, OSSINING, NY (US); EMMA HOLDRICH SCHWAGER, ENGLEWOOD, CO (US); STEPHANIE LANIUS, CAMBRIDGE, MA (US)

(21) Appl. No.: 16/854,281

(22) Filed: Apr. 21, 2020

Related U.S. Application Data
(60) Provisional application No. 62/838,979, filed on Apr. 26, 2019.

(51) Int. Cl. G01N 33/70; G01N 33/493

(52) U.S. Cl. CPC G01N 33/70; G01N 33/493; G01N 2800/56; G01N 2800/50; G01N 2800/347

(57) ABSTRACT

A method and system method for determining a patient's acute kidney injury (AKI) stage, including: receiving a patient's current AKI stage; calculating a patient's new AKI stage; comparing the new AKI stage to the current AKI stage; updating the patient's AKI stage to the new AKI stage when the new AKI stage is greater than the current AKI stage; calculating AKI stage exit criteria and an AKI exit stage value; determining whether the AKI stage exit criteria are satisfied; and reducing the patient's AKI stage to the exit AKI stage when the AKI stage exit criteria are satisfied.

### TECHNICAL FIELD

Various exemplary embodiments disclosed herein relate generally to a modification to staging guidelines for improved acute kidney injury (AKI) recovery detection.

### BACKGROUND

There are explicit medical guidelines (for example, Kidney Disease Improving Global Outcomes (KDIGO) criteria) to indicate if a patient had acute kidney injury. However, those guidelines are usually used retrospectively and designed to detect AKI, but not describe AKI over time. Furthermore, there are no guidelines specifying when AKI ended. Defining recovery of AKI as the moment when no AKI criteria is satisfied is problematic as many patients fluctuate between stages using the existing criteria. For example, a patient may be borderline between stage 1 and stage 0 (no AKI), and thus oscillate between the two states. As a result, it is difficult to tell when the patient has finally recovered from AKI.

### SUMMARY

A summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

Various embodiments relate to a method for determining a patient's acute kidney injury (AKI) stage, including: receiving a patient's current AKI stage; calculating a patient's new AKI stage; comparing the new AKI stage to the current AKI stage; updating the patient's AKI stage to the new AKI stage when the new AKI stage is greater than the current AKI stage; calculating AKI stage exit criteria and an AKI exit stage value; determining whether the AKI stage exit criteria are satisfied; and reducing the patient's AKI stage to the exit AKI stage when the AKI stage exit criteria are satisfied.

Various embodiments are described, wherein patient's AKI stage remains unchanged when the AKI stage exit criteria are not satisfied.

Various embodiments are described, wherein calculating the patients new AKI stage includes: receiving the patient's weight and periodic urine output; and comparing all normalized periodic urine output over a plurality of time periods to a corresponding plurality of urine output thresholds.

Various embodiments are described, wherein calculating the patients new AKI stage includes: receiving the patient's weight and periodic urine output; setting the patient's AKI stage to stage 3 when all the patients normalized periodic urine output in the last 24 hours<0.3 ml/dL or the patient's total urine output in the last 12 hours<50 ml; setting the patient's AKI stage to stage 2 when all the patients normalized periodic urine output in the last 12 hours<0.5 ml/dL; and setting the patient's AKI stage to stage 1 when all the patients normalized periodic urine output in the last 6 hours<0.5 ml/dL.

Various embodiments are described, wherein calculating AKI stage exit criteria and an AKI exit stage value includes: receiving the patient's weight and periodic urine output;

calculating the average normalized periodic urine output over a plurality of time periods; and comparing the calculated average normalized periodic urine output values over a plurality of time periods to a corresponding plurality of urine output thresholds.

Various embodiments are described, wherein calculating AKI stage exit criteria and an AKI exit stage value includes: receiving the patient's weight and periodic urine output; calculating a first average periodic urine output in the last 24 hours; setting the patient's AKI exit stage to stage 3 when the first average periodic urine output in the last 24 hours<0.3 ml/dL or the patient's total urine output in the last 12 hours<50 ml; calculating a second average periodic urine output in the last 12 hours; setting the patient's AKI stage to stage 2 when the second average periodic urine output in the last 12 hours<0.5 ml/dL; calculating a third average hourly urine output in the last 26 hours; and setting the patient's AKI stage to stage 1 when the third average periodic urine output in the last 6 hours<0.5 ml/dL.

Various embodiments are described, wherein calculating the patients new AKI stage includes: receiving the patient's weight and periodic urine output; comparing all normalized periodic urine output over a plurality of time periods to a corresponding set of urine output thresholds; calculating AKI stage exit criteria and an AKI exit stage value includes: calculating the average normalized periodic urine output over a plurality of time periods; and comparing the calculated average normalized periodic urine output values over a plurality of time periods to a corresponding set of urine output thresholds.

Further various embodiments relate to a system configured to predict the next location for a patient in a healthcare facility, including: a memory; a processor coupled to the memory, wherein the processor is configured to: receive a patient's current AKI stage; calculate a patient's new AKI stage; compare the new AKI stage to the current AKI stage; update the patient's AKI stage to the new AKI stage when the new AKI stage is greater than the current AKI stage; calculate AKI stage exit criteria and an AKI exit stage value; determine whether the AKI stage exit criteria are satisfied; and reduce the patient's AKI stage to the exit AKI stage when the AKI stage exit criteria are satisfied.

The system of claim 8, wherein patient's AKI stage remains unchanged when the AKI stage exit criteria are not satisfied.

Various embodiments are described, wherein calculating the patients new AKI stage includes: receiving the patient's weight and periodic urine output; and comparing all normalized periodic urine output over a plurality of time periods to a corresponding plurality of urine output thresholds.

Various embodiments are described, wherein calculating the patients new AKI stage includes: receiving the patient's weight and periodic urine output; setting the patient's AKI stage to stage 3 when all the patients normalized periodic urine output in the last 24 hours<0.3 ml/dL or the patient's total urine output in the last 12 hours<50 ml; setting the patient's AKI stage to stage 2 when all the patients normalized periodic urine output in the last 12 hours<0.5 ml/dL; and setting the patient's AKI stage to stage 1 when all the patients normalized periodic urine output in the last 6 hours<0.5 ml/dL.

Various embodiments are described, wherein calculating AKI stage exit criteria and an AKI exit stage value includes: receiving the patient's weight and periodic urine output; calculating the average normalized periodic urine output over a plurality of time periods; and comparing the calculated average normalized periodic urine output values over a plurality of time periods to a corresponding plurality of urine output thresholds.

Various embodiments are described, wherein calculating AKI stage exit criteria and an AKI exit stage value includes: receiving the patient's weight and periodic urine output; calculating a first average periodic urine output in the last 24 hours; setting the patient's AKI exit stage to stage 3 when the first average periodic urine output in the last 24 hours<0.3 ml/dL or the patient's total urine output in the last 12 hours<50 ml; calculating a second average periodic urine output in the last 12 hours; setting the patient's AKI stage to stage 2 when the second average periodic urine output in the last 12 hours<0.5 ml/dL; calculating a third average hourly urine output in the last 26 hours; and setting the patient's AKI stage to stage 1 when the third average periodic urine output in the last 6 hours<0.5 ml/dL.

Various embodiments are described, wherein calculating the patients new AKI stage includes: receiving the patient's weight and periodic urine output; comparing all normalized periodic urine output over a plurality of time periods to a corresponding set of urine output thresholds; calculating AKI stage exit criteria and an AKI exit stage value includes: calculating the average normalized periodic urine output over a plurality of time periods; and comparing the calculated average normalized periodic urine output values over a plurality of time periods to a corresponding set of urine output thresholds.

Further various embodiments relate to a non-transitory machine-readable storage medium encoded with instructions for determining a patient's acute kidney injury (AKI) stage, including: instructions for receiving a patient's current AKI stage; instructions for calculating a patient's new AKI stage; instructions for comparing the new AKI stage to the current AKI stage; instructions for updating the patient's AKI stage to the new AKI stage when the new AKI stage is greater than the current AKI stage; instructions for calculating AKI stage exit criteria and an AKI exit stage value; instructions for determining whether the AKI stage exit criteria are satisfied; and instructions for reducing the patient's AKI stage to the exit AKI stage when the AKI stage exit criteria are satisfied.

Various embodiments are described, wherein patient's AKI stage remains unchanged when the AKI stage exit criteria are not satisfied.

Various embodiments are described, wherein instructions for calculating the patients new AKI stage includes: instructions for receiving the patient's weight and periodic urine output; and instructions for comparing all normalized periodic urine output over a plurality of time periods to a corresponding plurality of urine output thresholds.

Various embodiments are described, wherein instructions for calculating the patients new AKI stage includes: instructions for receiving the patient's weight and periodic urine output; instructions for setting the patient's AKI stage to stage 3 when all the patients normalized periodic urine output in the last 24 hours<0.3 ml/dL or the patient's total urine output in the last 12 hours<50 ml; instructions for setting the patient's AKI stage to stage 2 when all the patients normalized periodic urine output in the last 12 hours<0.5 ml/dL; and instructions for setting the patient's AKI stage to stage 1 when all the patients normalized periodic urine output in the last 6 hours<0.5 ml/dL.

Various embodiments are described, wherein instructions for calculating AKI stage exit criteria and an AKI exit stage value includes: instructions for receiving the patient's weight and periodic urine output; instructions for calculating the average normalized periodic urine output over a plurality of time periods; and instructions for comparing the calculated average normalized periodic urine output values over a plurality of time periods to a corresponding plurality of urine output thresholds.

Various embodiments are described, wherein instructions for calculating AKI stage exit criteria and an AKI exit stage value includes: instructions for receiving the patient's weight and periodic urine output; instructions for calculating a first average periodic urine output in the last 24 hours; instructions for setting the patient's AKI exit stage to stage 3 when the first average periodic urine output in the last 24 hours<0.3 ml/dL or the patient's total urine output in the last 12 hours<50 ml; instructions for calculating a second average periodic urine output in the last 12 hours; instructions for setting the patient's AKI stage to stage 2 when the second average periodic urine output in the last 12 hours<0.5 ml/dL; instructions for calculating a third average hourly urine output in the last 26 hours; and instructions for setting the patient's AKI stage to stage 1 when the third average periodic urine output in the last 6 hours<0.5 ml/dL.

Various embodiments are described, wherein instructions for calculating the patients new AKI stage includes: instructions for receiving the patient's weight and periodic urine output; instructions for comparing all normalized periodic urine output over a plurality of time periods to a corresponding set of urine output thresholds; instructions for calculating AKI stage exit criteria and an AKI exit stage value includes: instructions for calculating the average normalized periodic urine output over a plurality of time periods; and instructions for comparing the calculated average normalized periodic urine output values over a plurality of time periods to a corresponding set of urine output thresholds.

### BRIEF DESCRIPTION OF THE DRAWINGS

In order to better understand various exemplary embodiments, reference is made to the accompanying drawings, wherein:

FIG. 1 illustrates the AKIN guidelines and how they are evaluated;

FIG. 2 shows two plots of the UO stage plotted over time;

FIG. 3 illustrates a flowchart showing the determination of the current AKI stage;

FIG. 4 illustrates a flowchart showing the determination of whether the patient's AKI stage should be reduced;

FIG. 5 illustrates a method for updating the patient's AKI stage as needed; and

FIG. 6 illustrates an exemplary hardware diagram for implementing the system and method described herein.

To facilitate understanding, identical reference numerals have been used to designate elements having substantially the same or similar structure and/or substantially the same or similar function.

### DETAILED DESCRIPTION

The description and drawings illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments.

Acute kidney injury (AKI) is an ICU-acquired illness that occurs in a significant majority of critically ill patients. AKI may be caused by multiple conditions such as sepsis, infection, drug nephrotoxicity, surgery, and other idiopathic causes. Because AKI is a secondary condition, early signs of AKI can be easily missed leading to progressively severe forms of kidney injury. Patients who develop AKI in the ICU have longer ICU and hospital stays, higher mortality, and worse outcomes post-discharge. Besides these problems, AKI has a significant financial impact on the health care system and patients.

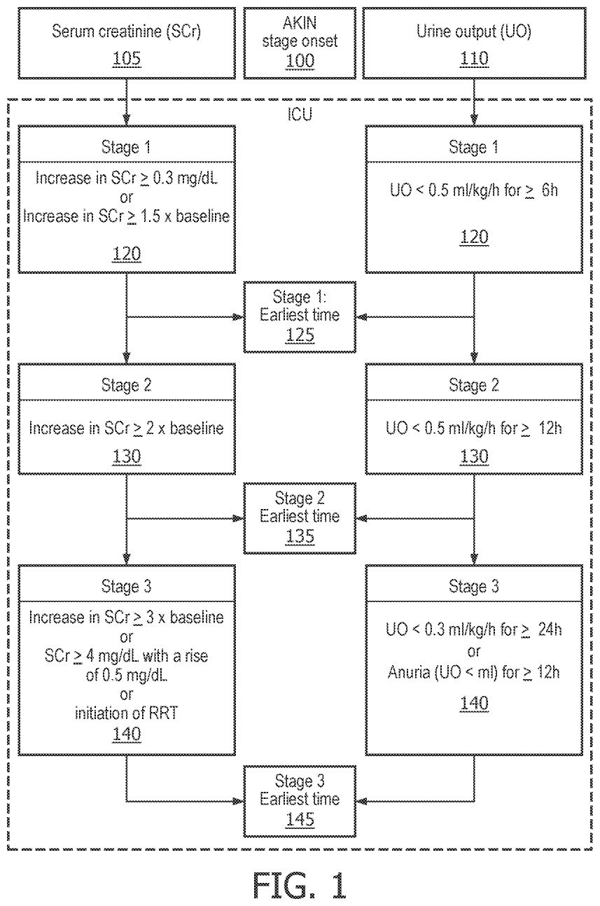

To monitor and diagnose AKI, the Acute Kidney Injury Network (AKIN) developed a set of guidelines to stage AKI. FIG. 1 illustrates these guidelines and how they are evaluated. These guidelines 100 use urine output (UO) 110 and serum creatinine (SCr) values 105 to classify AKI risk into 3 stages: Stage 1 120, low risk of AKI; Stage 2 130, moderate risk of AKI; Stage 3 140, severe risk of AKI. Specifically, Stage 1 for SCr is entered when the increase in SCr≥0.3 mg/dL or ≥1.5× baseline SCr. Stage 2 for SCr is entered when the increase in SCr≥2× baseline SCr. Stage 3 for SCr is entered when the increase in SCr≥3× baseline SCr or SCr≥4.0 mg/dL with a rise of 0.5 mg/dL or initiation of renal replacement therapy (RRT). Further, stage 1 for UO is entered when UO<0.5 ml/kg/h for ≥6 hours. Stage 2 for UO is entered when UO<0.5 ml/kh/h for ≥12 hours. Stage 3 for UO is entered when UO<0.3 ml/kg/h for >24 hours or Anuria (UO<50 ml) for ≥12 hours. These guidelines were designed to be used by clinicians to manually evaluate AKI risk. Moreover, these guidelines are better suited for retrospective analysis of patient records. Further, the earliest detections of stage 1 AKI 125, stage 2 AKI 135, and stage 3 AKI 145 occur only when both the SCr and UO reach those stages as shown.

To be able to apply this algorithm in real-time, these guidelines were modified in collaboration with the Mayo clinic. The modified guidelines were validated using clinician annotated data. See Development and validation of electronic surveillance tool for acute kidney injury: A retrospective analysis, A. Ahmed et al., J Crit Care. 2015 October, 30(5):988-93. doi: 10.1016/j.jcrc.2015.05.007. Epub 2015 May 19.

There are explicit medical guidelines (for example, KDIGO criteria) to indicate if a patient had an acute kidney injury. However, those guidelines are usually used retrospectively and designed to detect AKI, but not to describe AKI over time. The embodiments described herein detect how long AKI persisted. Furthermore, there are no guidelines specifying when AKI ended. Defining recovery from AKI as the moment when no AKI criteria is satisfied is problematic as many patients fluctuate between stages during their illness. The method described herein is a sophisticated method to avoid those fluctuations. The method recognizes that entering and leaving an AKI stage are very different.

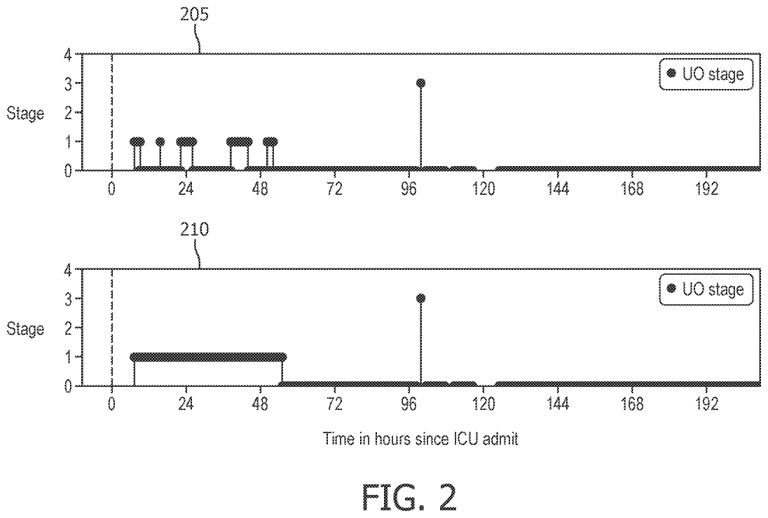

The KDIGO guidelines previously have been converted into an electronic algorithm for real-time AKI risk assessment. However, there are still some challenges in meaningful representation of AKI risk. Using the current guidelines, the risk from staging using urine output can be noisy. FIG. 2 shows two plots of the UO stage plotted over time. The top plot shows the UO stage according the current guidelines versus time in hours since the patient was admitted to the ICU. As can be seen in the first 60 hours, the patients UO stage oscillates between stage 0 and stage 1. Because changes in renal function occur over longer time periods, these transient increases and decreases in AKI stage indicated by urine output do not accurately represent AKI risk. Therefore, many researchers choose to ignore the urine staging part for evaluating AKI risk, as it is difficult to implement and to interpret. As creatinine only increases sometime after kidney injury has occurred, urine output may be an earlier indicator, which is really valuable in preventing, treating, and minimizing AKI. Therefore, there is an advantage in using both criteria.

However, there is no unambiguous interpretation of the urine staging part. This is due to different problems with urine staging. For example, urine chartings are usually not done automatically. As a result, the UO data is not consistently and reliably collected. Further, these irregular urine chartings result in irregular UO stage changes. Currently, there is no medical interpretation of stage fluctuations and this information is mostly confusing to physicians.

Detecting when a patient gets better regarding their kidneys may also add valuable information to the treatment of the patient. For example, nephrotoxic medications are often discontinued in patients with AKI. These medications may be resumed when the AKI risk goes down. However, to reliably use AKI staging, the fluctuations based on urine charting should be removed. With an accurate continuous AKI stage determination not only representing kidney injury, but also the recovery, physicians will be able to better decide how to continue treatment, for example, when is is safe to start administering nephrotoxic medications again. KDIGO criteria are useful to define if a patient had AKI or not, as well as determining when AKI first occurs for the patient.

An embodiment of a methods for implementing the KDIGO criteria that smoothes out the urine staging will now be described that provides valuable information the treating physicians. Also, a minimum number of measurements criteria may detect if urine was not charted or if no urine was produced.

An embodiment of a practical method and implementation on how to continuously assess AKI stages will now be described. The key insight in this method is that changes in renal physiology take a long time to (many hours to days) to manifest. So a patient's AKI risk is unlikely to change hourly. To implement this in practice, a modification is proposed to the current guidelines, whereby the criteria for increasing the AKI stage remains the same as before, but the criteria for measuring recovery (i.e., decreasing the AKI stage) is made stricter. As a result, once a patient has a specific stage of AKI, using the new method, it will be more difficult to go to a lower (or no) AKI risk than using the standard AKI stage definitions. This results in the patient being in a higher AKI stage longer, but any decrease in AKI stage is likely to be indicative of true recovery.

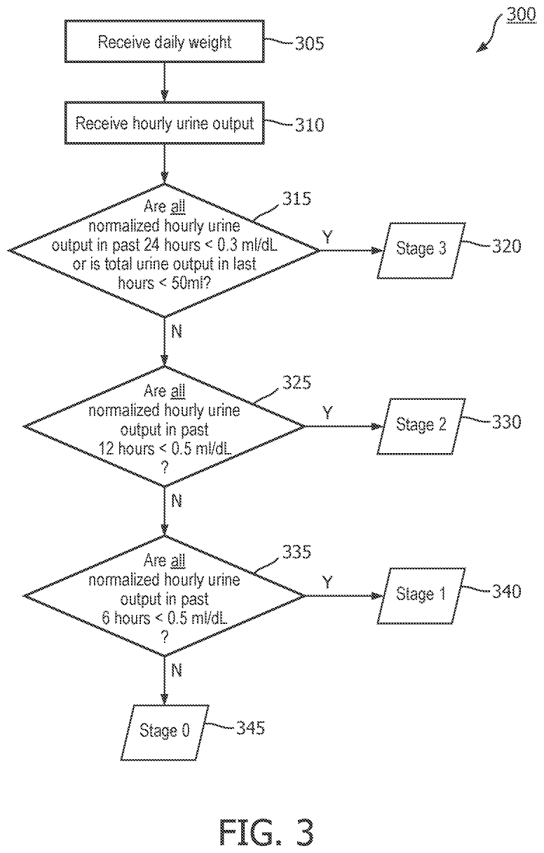

FIG. 3 illustrates a flowchart showing the determination of the current AKI stage. First the method 300 receives the patient's daily weight 305 and hourly urine outputs 310. Next, the method 300 determines 315 if all of the normalized hourly urine output in the past 24 hours<0.3 ml/dL or if the total urine output in the last 12 hours is <50 ml. If yes, then the method determines that the patient is in UO stage 3 320. If not, the method 300 determines 325 if all the normalized hourly urine output in the past 12 hours<0.5 ml/dL. If yes, then the method determines that the patient is in UO stage 2 330. If not, the method 300 determines 335 if all the normalized hourly urine output in the past 6 hours<0.5 ml/dL. If yes, then the method determines that the patient is in UO stage 1 340. If no, then the method determines that the patient is in UO stage 0 350. The method of FIG. 3 uses specific values of urine output and time lengths, but each of these values may be different values. As more and more patients are evaluated or as patients may be determined to be in different groups, the urine output threshold values and the time thresholds may be different from those above. Further, the established guidelines for AKI may evolve. Also, the urine output threshold values may be the same or different.

Note that the algorithm to determine the current AKI stage using urine output uses the maximum urine output over a 6, 12, or 24 hour period to be below a threshold for the AKI stage to be 1, 2 or 3 respectively. This makes it difficult for a patient's risk to be high. Note that if these same criteria are used to evaluate a decrease in AKI risk, then a single high value of urine output can lower a patient's AKI risk. As discussed above, the patient's risk for AKI varies slowly over time.

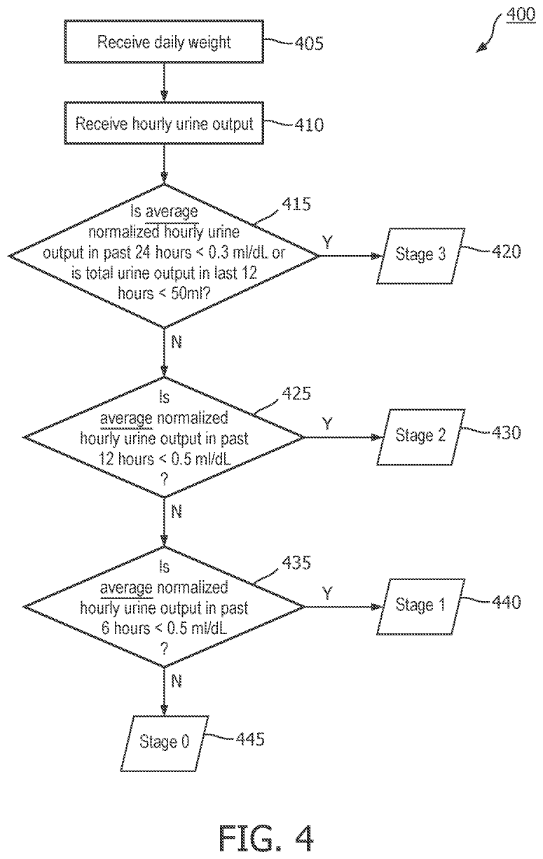

To prevent this, a new set of criteria for AKI recovery (i.e., decreasing the AKI stage) uses the average urine output over a 6, 12, or 24 hour period which is compared to the UO threshold for the patient's AKI stage to reduce from Stage 1, 2 or 3 respectively. FIG. 4 illustrates a flowchart showing the determination of whether the patient's AKI stage should be reduced. First the method 400 receives the patient's daily weight 405 and hourly urine outputs 410. Next, the method 400 calculates the average normalized hour urine output over the last 24 hours and determines 415 if the average normalized hourly urine output in the past 24 hours<0.3 ml/dL or if the total urine output in the last 12 hours is <50. If yes, then the method determines that the patient is in UO stage 3 420. If not, the method 400 calculates the average normalized hour urine output over the last 12 hours and determines 425 if the average normalized hourly urine output in the past 12 hours<0.5 ml/dL. If yes, then the method determines that the patient is in UO stage 2 430. If not, the method 400 the method 400 calculates the average normalized hour urine output over the last 6 hours and determines 435 if the average normalized hourly urine output in the past 6 hours<0.5 ml/dL. If yes, then the method determines that the patient is in UO stage 1 440. If no, then the method determines that the patient is in UO stage 0 450. The method of FIG. 4 uses specific values of urine output and time lengths, but each of these values may be different values. As more and more patients are evaluated or as patients may be determined to be in different groups, the urine output threshold values and the time thresholds may be different from those above. Further, the established guidelines for AKI may evolve. Also, the urine output threshold values may be the same or different. Further, in the examples shown in FIGS. 3 and 4, the threshold values and time values are shown as being the same for the different stages, but they could be different from one another as well.

Also, the methods 300 and 400 show the calculation of four AKI stages, i.e., 0, 1, 2, and 3. If the AKI guidelines change, more for fewer stages may be calculated as well. The methods of 300 and 400 would easily be adapted to increase or decrease the number of AKI stages, to accommodate changes in the AKI guidelines.

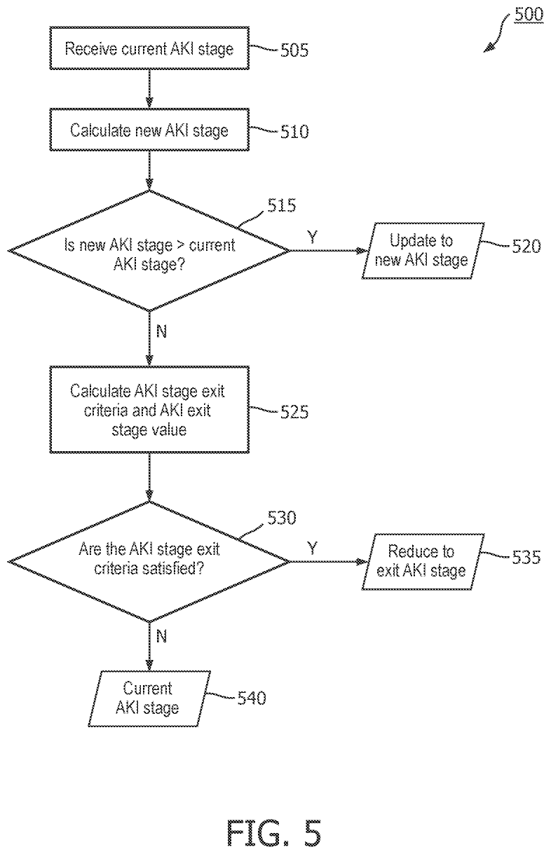

FIG. 5 illustrates a method for updating the patient's AKI stage as needed. The method 500 begins by receiving the current AKI stage for the patient 505. The method then calculates 510 a new AKI stage for the patient using the method 300 shown in FIG. 3. The method 500 then determines if the new AKI stage is greater than the current AKI stage 515. If so, the patient's AKI stage is updated to the new AKI stage 520. If not, the method 500 calculates 525 an AKI exit stage using the AKI stage exit criteria as implement in method 400 as shown in FIG. 4. Next, the method determines if the AKI exit stage criteria are satisfied 530. If so, the patient's AKI stage is updated/reduced to the exit stage value 535. If not, the patient's AKI stage remains unchanged.

This means, in order for AKI stage to increase, it is sufficient that maximum UO is lower than the threshold. However, in order for AKI stage to decrease, both maximum UO and average UO have to increase above the threshold. This results in the same detection in increased AKI risk as before, but now the decrease in AKI stage during recovery is changed. This approach, for example, prevents a small number of measurements from causing the AKI stage to fluctuate quickly.

An example of staging over time using this method is shown in the lower plot of FIG. 2. The top figure shows the AKI stage using the method of FIG. 1. From Hour 6 to Hour 50, the patient's AKI stage fluctuates between 0 and 1 multiple times. Sometimes these changes occur within a couple of hours. However, kidney injury occurs over longer time-scales so this does not reflect true physiology. The bottom figure shows the AKI stage using the method 500 shown in FIG. 5. As shown, once the patients AKI stage reaches 1, it stays there without fluctuation because of the exit criteria applied by the method 400. This results in an AKI stage determination that is clinically more meaningful and actionable.

The methods described herein may be used in various ways. They can help to detect when it is safe again to administer a nephrotoxic medication again. This can help physicians to objectify their decisions. Another application of these methods is in retrospectively automatically annotating recovery. This is critical in modelling AKI recovery. This will be powerful in combination with the AKI detection as it gives a good overview about the patient status. The applications are diverse, from giving guidance to physicians if the status of the patient ameliorated to predicting if a certain treatment will allow the patient health status to improve.

The various methods above receive measured patient data and determine the patients current AKI stage including determining whether the patients AKI stage should be lowered. Different methods are used to increase and decrease a patients AKI stage. Such methods use various calculations and comparisons to provide a practical application of the method in determining the patients AKI stage, which then may be used by a caregiver to make various treatment and care decisions for the patient. The current methods of continuously calculating the patient's AKI stage may result in quick fluctuations in the patient's AKI stage that do not accurately reflect the patient's AKI condition, which typically changes slowly, hence the recovery from AKI is typically slower then that which is indicated by the quick oscillation in AKI values as calculated by current methods. Further, the methods described herein reduce the risk that the physician prematurely determines that the patient's AKI risk and status has reduced enough to resume treatments that had to cease due to the potential for AKI. Such practical application provides new insights to physicians treating patients that may be experiencing AKI.

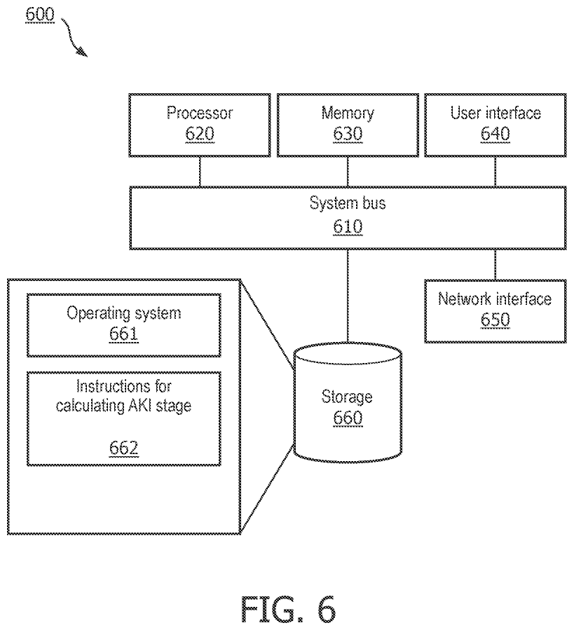

FIG. 6 illustrates an exemplary hardware diagram 600 for implementing the system and method described above. The exemplary hardware 600 may implement the methods of FIGS. 3, 4, and 5. As shown, the device 600 includes a processor 620, memory 630, user interface 640, network interface 650, and storage 660 interconnected via one or more system buses 610. It will be understood that FIG. 6 constitutes, in some respects, an abstraction and that the actual organization of the components of the device 600 may be more complex than illustrated.

The processor 620 may be any hardware device capable of executing instructions stored in memory 630 or storage 660 or otherwise processing data. As such, the processor may include a microprocessor, field programmable gate array (FPGA), application-specific integrated circuit (ASIC), or other similar devices.

The memory 630 may include various memories such as, for example L1, L2, or L3 cache or system memory. As such, the memory 630 may include static random-access memory (SRAM), dynamic RAM (DRAM), flash memory, read only memory (ROM), or other similar memory devices.

The user interface 640 may include one or more devices for enabling communication with a user. For example, the user interface 640 may include a display, a touch interface, a mouse, and/or a keyboard for receiving user commands. In some embodiments, the user interface 640 may include a command line interface or graphical user interface that may be presented to a remote terminal via the network interface 650.

The network interface 650 may include one or more devices for enabling communication with other hardware devices. For example, the network interface 650 may include a network interface card (NIC) configured to communicate according to the Ethernet or other communications protocols, including wireless protocols. Additionally, the network interface 650 may implement a TCP/IP stack for communication according to the TCP/IP protocols. Various alternative or additional hardware or configurations for the network interface 650 will be apparent.

The storage 660 may include one or more machine-readable storage media such as read-only memory (ROM), random-access memory (RAM), magnetic disk storage media, optical storage media, flash-memory devices, or similar storage media. In various embodiments, the storage 660 may store instructions for execution by the processor 620 or data upon with the processor 620 may operate. For example, the storage 660 may store a base operating system 661 for controlling various basic operations of the hardware 600. The storage may also store instructions for carrying out the methods of FIGS. 3, 4, and 5 and the other methods described above.

It will be apparent that various information described as stored in the storage 660 may be additionally or alternatively stored in the memory 630. In this respect, the memory 630 may also be considered to constitute a “storage device” and the storage 660 may be considered a “memory.” Various other arrangements will be apparent. Further, the memory 630 and storage 660 may both be considered to be “non-transitory machine-readable media.” As used herein, the term “non-transitory” will be understood to exclude transitory signals but to include all forms of storage, including both volatile and non-volatile memories.

While the host device 600 is shown as including one of each described component, the various components may be duplicated in various embodiments. For example, the processor 620 may include multiple microprocessors that are configured to independently execute the methods described herein or are configured to perform steps or subroutines of the methods described herein such that the multiple processors cooperate to achieve the functionality described herein.

As described above, the embodiments described herein may be implemented as software running on a processor with an associated memory and storage. The processor may be any hardware device capable of executing instructions stored in memory or storage or otherwise processing data. As such, the processor may include a microprocessor, field programmable gate array (FPGA), application-specific integrated circuit (ASIC), graphics processing units (GPU), specialized neural network processors, cloud computing systems, or other similar devices.

The memory may include various memories such as, for example L1, L2, or L3 cache or system memory. As such, the memory may include static random-access memory (SRAM), dynamic RAM (DRAM), flash memory, read only memory (ROM), or other similar memory devices.

The storage may include one or more machine-readable storage media such as read-only memory (ROM), random-access memory (RAM), magnetic disk storage media, optical storage media, flash-memory devices, or similar storage media. In various embodiments, the storage may store instructions for execution by the processor or data upon with the processor may operate. This software may implement the various embodiments described above.

Further such embodiments may be implemented on multiprocessor computer systems, distributed computer systems, and cloud computing systems. For example, the embodiments may be implemented as software on a server, a specific computer, on a cloud computing, or other computing platform.

Any combination of specific software running on a processor to implement the embodiments of the invention, constitute a specific dedicated machine.

As used herein, the term “non-transitory machine-readable storage medium” will be understood to exclude a transitory propagation signal but to include all forms of volatile and non-volatile memory.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. A method for determining a patient's acute kidney injury (AK) stage, comprising:
receiving a patient's current AKI stage;
calculating a patient's new AKI stage;
comparing the new AKI stage to the current AKI stage;
updating the patient's AKI stage to the new AKI stage when the new AKI stage is greater than the current AKI stage;
calculating AKI stage exit criteria and an AKI exit stage value;
determining whether the AKI stage exit criteria are satisfied; and
reducing the patient's AKI stage to the exit AKI stage when the AKI stage exit criteria are satisfied.

2. The method of claim 1, wherein patient's AKI stage remains unchanged when the AKI stage exit criteria are not satisfied.

3. The method of claim 1, wherein calculating the patients new AKI stage includes:
receiving the patient's weight and periodic urine output; and
comparing all normalized periodic urine output over a plurality of time periods to a corresponding plurality of urine output thresholds.

4. The method of claim 1, wherein calculating the patients new AKI stage includes:
receiving the patient's weight and periodic urine output;
setting the patient's AKI stage to stage 3 when all the patients normalized periodic urine output in the last 24 hours<0.3 ml/dL or the patient's total urine output in the last 12 hours<50 ml;
setting the patient's AKI stage to stage 2 when all the patients normalized periodic urine output in the last 12 hours<0.5 ml/dL; and
setting the patient's AKI stage to stage 1 when all the patients normalized periodic urine output in the last 6 hours<0.5 ml/dL.

5. The method of claim 1, wherein calculating AKI stage exit criteria and an AKI exit stage value includes:
receiving the patient's weight and periodic urine output;
calculating the average normalized periodic urine output over a plurality of time periods; and
comparing the calculated average normalized periodic urine output values over a plurality of time periods to a corresponding plurality of urine output thresholds.

6. The method of claim 1, wherein calculating AKI stage exit criteria and an AKI exit stage value includes:
receiving the patient's weight and periodic urine output;
calculating a first average periodic urine output in the last 24 hours;
setting the patient's AKI exit stage to stage 3 when the first average periodic urine output in the last 24 hours<0.3 ml/dL or the patient's total urine output in the last 12 hours<50 ml;
calculating a second average periodic urine output in the last 12 hours;
setting the patient's AKI stage to stage 2 when the second average periodic urine output in the last 12 hours<0.5 ml/dL;
calculating a third average hourly urine output in the last 26 hours; and
setting the patient's AKI stage to stage 1 when the third average periodic urine output in the last 6 hours<0.5 ml/dL.

7. The method of claim 1, wherein
calculating the patients new AKI stage includes:
receiving the patient's weight and periodic urine output;
comparing all normalized periodic urine output over a plurality of time periods to a corresponding set of urine output thresholds;
calculating AKI stage exit criteria and an AKI exit stage value includes:
calculating the average normalized periodic urine output over a plurality of time periods; and
comparing the calculated average normalized periodic urine output values over a plurality of time periods to a corresponding set of urine output thresholds.

8. A system configured to predict the next location for a patient in a healthcare facility, comprising:
a memory;
a processor coupled to the memory, wherein the processor is configured to:
receive a patient's current AKI stage;
calculate a patient's new AKI stage;
compare the new AKI stage to the current AKI stage;
update the patient's AKI stage to the new AKI stage when the new AKI stage is greater than the current AKI stage;
calculate AKI stage exit criteria and an AKI exit stage value;
determine whether the AKI stage exit criteria are satisfied; and
reduce the patient's AKI stage to the exit AKI stage when the AKI stage exit criteria are satisfied.

9. The system of claim 8, wherein patient's AKI stage remains unchanged when the AKI stage exit criteria are not satisfied.

10. The system of claim 8, wherein calculating the patients new AKI stage includes:
receiving the patient's weight and periodic urine output; and
comparing all normalized periodic urine output over a plurality of time periods to a corresponding plurality of urine output thresholds.

11. The system of claim 8, wherein calculating the patients new AKI stage includes:
receiving the patient's weight and periodic urine output;
setting the patient's AKI stage to stage 3 when all the patients normalized periodic urine output in the last 24 hours<0.3 ml/dL or the patient's total urine output in the last 12 hours<50 ml;
setting the patient's AKI stage to stage 2 when all the patients normalized periodic urine output in the last 12 hours<0.5 ml/dL; and
setting the patient's AKI stage to stage 1 when all the patients normalized periodic urine output in the last 6 hours<0.5 ml/dL.

12. The system of claim 8, wherein calculating AKI stage exit criteria and an AKI exit stage value includes:
receiving the patient's weight and periodic urine output;
calculating the average normalized periodic urine output over a plurality of time periods; and
comparing the calculated average normalized periodic urine output values over a plurality of time periods to a corresponding plurality of urine output thresholds.

13. The system of claim 8, wherein calculating AKI stage exit criteria and an AKI exit stage value includes:
receiving the patient's weight and periodic urine output;
calculating a first average periodic urine output in the last 24 hours;
setting the patient's AKI exit stage to stage 3 when the first average periodic urine output in the last 24 hours<0.3 ml/dL or the patient's total urine output in the last 12 hours<50 ml;
calculating a second average periodic urine output in the last 12 hours;
setting the patient's AKI stage to stage 2 when the second average periodic urine output in the last 12 hours<0.5 ml/dL;
calculating a third average hourly urine output in the last 26 hours; and
setting the patient's AKI stage to stage 1 when the third average periodic urine output in the last 6 hours<0.5 ml/dL.

14. The system of claim 8, wherein
calculating the patients new AKI stage includes:
receiving the patient's weight and periodic urine output;
comparing all normalized periodic urine output over a plurality of time periods to a corresponding set of urine output thresholds;
calculating AKI stage exit criteria and an AKI exit stage value includes:
calculating the average normalized periodic urine output over a plurality of time periods; and
comparing the calculated average normalized periodic urine output values over a plurality of time periods to a corresponding set of urine output thresholds.

15. A non-transitory machine-readable storage medium encoded with instructions for determining a patient's acute kidney injury (AKI) stage, comprising:
instructions for receiving a patient's current AKI stage;
instructions for calculating a patient's new AKI stage;
instructions for comparing the new AKI stage to the current AKI stage;
instructions for updating the patient's AKI stage to the new AKI stage when the new AKI stage is greater than the current AKI stage;
instructions for calculating AKI stage exit criteria and an AKI exit stage value;
instructions for determining whether the AKI stage exit criteria are satisfied; and
instructions for reducing the patient's AKI stage to the exit AKI stage when the AKI stage exit criteria are satisfied.

16. The non-transitory machine-readable storage medium of claim 15, wherein patient's AKI stage remains unchanged when the AKI stage exit criteria are not satisfied.

17. The non-transitory machine-readable storage medium of claim 15, wherein instructions for calculating the patients new AKI stage includes:
instructions for receiving the patient's weight and periodic urine output; and
instructions for comparing all normalized periodic urine output over a plurality of time periods to a corresponding plurality of urine output thresholds.

18. The non-transitory machine-readable storage medium of claim 15, wherein instructions for calculating the patients new AKI stage includes:
instructions for receiving the patient's weight and periodic urine output;
instructions for setting the patient's AKI stage to stage 3 when all the patients normalized periodic urine output in the last 24 hours<0.3 ml/dL or the patient's total urine output in the last 12 hours<50 ml;
instructions for setting the patient's AKI stage to stage 2 when all the patients normalized periodic urine output in the last 12 hours<0.5 ml/dL; and
instructions for setting the patient's AKI stage to stage 1 when all the patients normalized periodic urine output in the last 6 hours<0.5 ml/dL.

19. The non-transitory machine-readable storage medium of claim 15, wherein instructions for calculating AKI stage exit criteria and an AKI exit stage value includes:
instructions for receiving the patient's weight and periodic urine output;
instructions for calculating the average normalized periodic urine output over a plurality of time periods; and
instructions for comparing the calculated average normalized periodic urine output values over a plurality of time periods to a corresponding plurality of urine output thresholds.

20. The non-transitory machine-readable storage medium of claim 15, wherein instructions for calculating AKI stage exit criteria and an AKI exit stage value includes:
instructions for receiving the patient's weight and periodic urine output;
instructions for calculating a first average periodic urine output in the last 24 hours;
instructions for setting the patient's AKI exit stage to stage 3 when the first average periodic urine output in the last 24 hours<0.3 ml/dL or the patient's total urine output in the last 12 hours<50 ml;
instructions for calculating a second average periodic urine output in the last 12 hours;
instructions for setting the patient's AKI stage to stage 2 when the second average periodic urine output in the last 12 hours<0.5 ml/dL;
instructions for calculating a third average hourly urine output in the last 26 hours; and
instructions for setting the patient's AKI stage to stage 1 when the third average periodic urine output in the last 6 hours<0.5 ml/dL.

21. The non-transitory machine-readable storage medium of claim 15, wherein
instructions for calculating the patients new AKI stage includes:
instructions for receiving the patient's weight and periodic urine output;
instructions for comparing all normalized periodic urine output over a plurality of time periods to a corresponding set of urine output thresholds;
instructions for calculating AKI stage exit criteria and an AKI exit stage value includes:
instructions for calculating the average normalized periodic urine output over a plurality of time periods; and
instructions for comparing the calculated average normalized periodic urine output values over a plurality of time periods to a corresponding set of urine output thresholds.
